(19) United States
(12) Patent Application Publication
(10) Pub. No.: US 2004/0110267 A1
(43) Pub. Date: Jun. 10, 2004

(54) Room temperature stable competent cells

(75) Inventor: Latha Sundar, San Diego, CA (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/675,113

(22) Filed: Sep. 30, 2003

Related U.S. Application Data
(63) Continuation-in-part of application No. 09/894,806, filed on Jun. 28, 2001.
(60) Provisional application No. 60/255,726, filed on Dec. 15, 2000.
(60) Provisional application No. 60/415,389, filed on Oct. 2, 2002.

(51) Int. Cl. C12N 1/20

(52) U.S. Cl. 435/252.1

(57) ABSTRACT

The invention relates to methods of producing storage-stable competent cells, preparations comprising such cells, and methods of using them. More particularly, the invention relates to improved methods of generating room-temperature stable dried competent cells by exposing cells to agents and conditions that increase the survival and better maintain the competence of dried competent cells. Improvements over prior art methods are achieved by modifications of the cell growth conditions, treatments of cells before, after, or during the induction of competence, modification of competence induction methods, and modifications to drying and post-drying process steps. The invention also provides kits comprising storage-stable competent cell preparations made according to the methods of the invention.

### RELATED APPLICATIONS

The present application is a continuation-in-part of U.S. Patent Application with Ser. No. 09/894,806, filed Jun. 28, 2001, which claims priority under 35 U.S.C. §119(e) to U.S. Provisional Application No. 60/255,726, filed Dec. 15, 2000. The present application also claims priority under 35 U.S.C. §119(e) to U.S. Provisional Application with serial No. 60/415,389, filed Oct. 2, 2002. Each of the above priority applications is hereby incorporated by reference in its entirety.

### FIELD OF THE INVENTION

The invention relates to competent cells which are stable at room temperature and to methods of generating such cells.

### BACKGROUND OF THE INVENTION

Cells which are primed for the uptake of nucleic acids are referred to as competent cells. These are cells which have been treated to make their cell membranes more permeable in order to facilitate the entry of exogenous nucleic acids. Competent cells serve as vehicles to store and amplify cloned sequences.

Typical methods of generating competent cells comprise growing cells to log phase or early stationary phase and exposing the cells to CaCl₂ at 0° C. (see, e.g., Sambrook, et al., In Molecular Cloning: a Laboratory Manual, 2nd Edition, eds. Sambrook, et al., Cold Spring Harbor Laboratory Press, (1989)). Other salts are also useful for rendering cells competent, e.g., RbCl₂ and hexamine cobalt chloride. Competent cells can be contacted immediately with exogenous DNA or frozen in glycerol or DMSO for subsequent use. Upon thawing to 4° C. and contacting with plasmid DNA, frozen competent cells typically have transformation efficiencies of 1×10⁵-1×10⁹ transformants/μg of plasmid DNA.

Temperatures of −80° C. and below have been used to preserve viability of competent cells (see, e.g., U.S. Pat. No. 4,981,797) since storage at higher temperatures is associated with rapid loss of viability and transformation efficiency within a period of days (see, e.g., Dagent, et al., Gene 6: 23-28 (1979) and Pope, et al., Nucl. Acids Res. 24(3): 536-537 (1996)). However, storage at −80° C. is problematic because of the high cost of equipment necessary to maintain this temperature. It is also difficult to ship competent cells and maintain their viability; generally, competent cells are shipped overnight on dry ice or in the presence of frozen packaging materials, under suboptimal conditions.

Attempts to store competent cells at higher temperatures have been described. U.S. Pat. No. 5,891,692, describes a method of storing competent bacterial cells at 20° C. to 4° C. without appreciably losing transformation efficiency or viability. The method relies on altering the fatty acid content of the bacteria and requires transforming bacterial cells with exogenous E. coli fabB genes.

Jessee, et al., WO 98/35018 disclose a method of lyophilizing competent cells to generate cells which are stable at −20° C. for up to a year. In this method, cells which have been previously frozen from −20° C. to −80° C. are lyophilized in the presence of a cryoprotectant. During lyophilization, the cells are exposed to a series of temperature steps from −45° C. to 10° C. at a rate of about 0.1° C. to 1.0° C./hour. Jessee, et al. report that the cells are stable at a range of temperatures, including room temperature. The competent cells are reported to retain transformation efficiencies of 1×10⁵ to 1×10⁹ transformants/μg of DNA.

There is a need in the art for highly competent storage-stable cells with increased stability at temperatures above −80° C., and particularly a need for such cells with increased stability at room temperature.

### SUMMARY OF THE INVENTION

The invention provides improved methods of generating storage-stable dried competent cells by exposing the cells to agents and conditions that increase the survival and better maintain the competence of dried competent cells. The basic steps for producing storage-stable competent cells include growing bacterial cells in culture medium, removing the medium and treating the cells to induce competence, and drying the cells. The invention encompasses modifications to each of these basic steps, such that both survival and transformation efficiency of re-constituted cells is dramatically improved over prior art methods.

In one aspect, the invention encompasses a method of generating storage-stable competent cells, the method comprising: a) growing bacterial cells in culture medium at hyperosmotic salt concentration; b) treating the cells to make them competent; c) contacting the cells with a solution comprising a reducing sugar, or a non-reducing sugar, or mixtures of both, thereby increasing the intracellular concentration of osmoprotective sugars within the cells; and d) drying the competent cells resulting from step (c) in the presence of a non-reducing sugar such that storage-stable competent cells are generated. In a preferred embodiment that the drying step is performed at a temperature above freezing.

In one embodiment the salt in the medium is NaCl. It is preferred that the NaCl is present in the culture medium at a concentration of 50-400 mM higher, preferably between 100 and 350 mM higher, more preferably between 150 mM to 225 mM higher, and most preferably about 200 mM higher than isoosmotic salt for the cell being grown.

In one embodiment, step (d) is performed with the competent cells at an initial concentration of 10⁹-10¹¹.

In another embodiment, the reducing sugar in step (c) is selected from the group consisting of fructose, glucose (dextrose), maltose, lactose, glucopyranose, ribose and cellobiose. In a preferred embodiment, the reducing sugar is fructose.

In another embodiment, the non-reducing sugar in step (c) is selected from trehalose, sucrose, sorbitol, α-methyl glucopyranoside, and α-methyl galactopyranoside.

In a preferred embodiment, the non-reducing sugar is sorbitol or α-methyl glucopyranoside.

In another embodiment, the solution in step (c) is a mixture of fructose and a non-reducing sugar selected from sorbitol or α-methyl glucopyranoside.

In another embodiment, the defined solution comprises the reducing sugar at a concentration of 10-25% (w/v).

In another embodiment, the bacterial cells are grown to a final OD₅₅₀ of 0.45 to 0.5 before step (b).

In another embodiment, step (b) comprises exposure to a chemical agent. In a preferred embodiment, the chemical agent is selected from the group consisting of CaCl₂, RbCl₂, MnCl₂, and hexamine cobalt chloride.

In another embodiment, the bacterial cells are Gram negative cells. In a preferred embodiment, the bacterial cells are Gram negative enteric cells.

In another embodiment, the culture medium comprises NaCl, casein hydrolysate and/or maltose.

In a preferred embodiment, the casein hydrolysate is present in the culture medium at a concentration of 11-15 g/liter, inclusive. It is further preferred that the casein hydrolysate is present in the culture medium at a concentration of 11-12 g/liter, inclusive. In another preferred embodiment, maltose is present in the culture medium at a concentration of 0.1-0.3% (w/v), inclusive. It is further preferred that maltose is present in the culture medium at a concentration of 0.2-0.3% (w/v), inclusive.

In another embodiment, the competent cells made according to the invention can be stored at temperatures above −80° C. for at least one month and maintain transformation efficiencies of at least 10⁵/μg DNA. In a preferred embodiment, the competent cells can be stored at temperatures of −20° C. or above for at least one month and maintain transformation efficiencies of at least 10⁵ μg DNA. It is further preferred that the competent cells can be stored at temperatures of 0° C. or above for at least one month and maintain transformation efficiencies of at least 10⁵/μg DNA. It is further preferred that the competent cells can be stored at temperatures of 4° C. or above for at least one month and maintain transformation efficiencies of at least 10⁵/μg DNA. It is further preferred that the competent cells can be stored at temperatures of 15° C. or above for at least one month and maintain transformation efficiencies of at least 10⁵/μg DNA. It is further preferred that the competent cells can be stored at temperatures of 20° C. or above for at least one month and maintain transformation efficiencies of at least 10⁵/μg DNA. Maintenance of higher transformation efficiencies at any of the aforementioned temperatures, e.g., maintenance of efficiency of at least 10⁶ colonies/μg DNA, is further preferred.

In another embodiment, the method further comprises the step, during or after step (d), of limiting the exposure of the competent cells to oxygen. It is preferred that the competent cells are stored in a vacuum stoppered vial. It is further preferred that the vial is stored in a sealed pouch. Limiting of the cells to exposure to oxygen can comprise of drying and/or storing the competent cells in the presence of an oxygen scavenger.

In another embodiment, the method further comprises the step, after step (d), of limiting exposure of the competent cells to moisture. In a preferred embodiment, the stopper in the stoppered vial is baked to remove moisture prior to use.

In another embodiment, the method further comprises the step, during or after step (d), of limiting the exposure of the competent cells to light. In a preferred embodiment, the limiting comprises storing the competent cells in a vial that has reduced transmittance of light.

The invention further encompasses a preparation of storage stable competent cells prepared according to the methods described herein.

The invention further encompasses a kit comprising a preparation of storage stable competent cells prepared according to the methods described herein above.

In another aspect, the invention encompasses a method of generating storage-stable competent cells, the method comprising: a) growing bacterial cells in culture medium at hyperosmotic salt concentration; b) removing the culture medium and treating the cells to make them competent, wherein the treating also comprises contacting the cells with an appropriate reducing or non-reducing sugar; and c) drying the competent cells resulting from step (b) in the presence of a non-reducing sugar, such that storage-stable competent cells are generated.

In one embodiment, the reducing sugar used in step (b) is selected from the group consisting of fructose, glucose (dextrose), maltose, lactose, glucopyranose, ribose and cellobiose. In a preferred embodiment, the reducing sugar is fructose.

In another embodiment, the defined solution comprises the reducing sugar at a concentration of 10-25% (w/v).

In another embodiment, the non-reducing sugar used in step (b) is selected from the group consisting of sucrose, melezitose, raffinose, α-methyl glucopyranoside, α-methyl galactopyranoside, etc. and sugar alcohol such as sorbitol, malitol, mannitol, etc.

In another embodiment, the bacterial cells are grown to a final OD₅₅₀ of 0.45 to 0.5 before step (b).

In a preferred embodiment, the non-reducing sugars are sorbitol and α-methyl glucopyranoside.

In another embodiment, step (b) comprises exposure to a chemical agent. In a preferred embodiment, the chemical agent is selected from the group consisting of CaCl₂, RbCl₂, MnCl₂, and hexamine cobalt chloride.

In another embodiment, the bacterial cells are Gram negative cells. In a preferred embodiment, the bacterial cells are Gram negative enteric cells.

In another embodiment, the culture medium comprises casein hydrolysate and/or maltose. In a preferred embodiment, casein hydrolysate is present in the culture medium at a concentration of 11-15 g/liter, inclusive. It is further preferred that casein hydrolysate is present in the culture medium at a concentration of 11-12 g/liter, inclusive. In another preferred embodiment, maltose is present in the culture medium at a concentration of 0.1-0.3% (w/v), inclusive. It is further preferred that the maltose is present in the culture medium at a concentration of 0.2-0.3% (w/v), inclusive.

In another embodiment, the competent cells made according to this aspect of the invention can be stored at temperatures above −80° C. for at least one month and maintain transformation efficiencies of at least 10⁵/μg DNA. In a preferred embodiment, the competent cells can be stored at temperatures of −20° C. or above for at least one month and maintain transformation efficiencies of at least 10⁵/μg DNA. It is further preferred that the competent cells can be stored at temperatures of 0° C. or above for at least one month and maintain transformation efficiencies of at least 10⁵/μg DNA. It is further preferred that the competent cells can be stored at temperatures of 4° C. or above for at least one month and maintain transformation efficiencies of at least 10⁵/μg DNA. It is further preferred that the competent cells can be stored at temperatures of 15° C. or above for at least one month and maintain transformation efficiencies of at least 10⁵/μg DNA. It is further preferred that the competent cells can be stored at temperatures of 20° C. or above for at least one month and maintain transformation efficiencies of at least 10⁵/μg DNA. Maintenance of higher transformation efficiencies at any of the aforementioned temperatures, e.g., maintenance of efficiency of at least 10⁶ colonies/μg DNA, is further preferred.

The invention further encompasses a preparation of storage stable competent cells prepared according to this aspect of the invention. The invention further encompasses a kit comprising such a preparation.

In another aspect, the invention encompasses a method of generating storage-stable competent cells, the method comprising: a) growing bacterial cells in culture medium; b) removing the culture medium and treating the cells to make them competent, wherein the treating comprises contacting the cells with a defined solution comprising one or both of proline and threonine; and c) drying the competent cells resulting from step (b) in the presence of a non-reducing sugar, such that storage-stable competent cells are generated.

In one embodiment, the defined solution comprises proline, threonine or both at a concentration of 0.5-7.5 mg/ml. In a preferred embodiment, the concentration of proline, threonine or both in the defined solution is from 2-4 mg/ml, inclusive.

The invention further encompasses a preparation of storage stable competent cells prepared according to this aspect of the invention. The invention further encompasses a kit comprising a preparation of storage stable competent cells prepared according to this aspect of the invention.

In another aspect, the invention encompasses a method of generating storage-stable competent cells, the method comprising: a) growing bacterial cells in culture medium at hyperosmotic salt concentration; b) treating the cells to make them competent; and c) contacting the cells with a solution comprising a reducing sugar or non-reducing sugar, or mixtures of both; and (d) drying the competent cells resulting from step (c) in the presence of a non-reducing sugar and gelatin, such that storage-stable competent cells are generated.

In one embodiment, the gelatin is present at 0.5 to 2.5% In a preferred embodiment, the gelatin is present at 0.8 to 1.2%.

In another aspect, the invention encompasses a method of producing a transformed cell, the method comprising a) obtaining cells generated according to any of the methods described herein; b) re-hydrating the cells; c) contacting the cells with a nucleic acid vector; and d) growing the cells, such that a transformed cell is produced.

In another aspect, the invention encompasses a method of producing a recombinant polypeptide comprising: a) obtaining cells generated according to any of the methods described herein; b) rehydrating the cells; c) contacting the cells with a nucleic acid vector encoding the recombinant polypeptide; and d) growing the cells in a cell growth medium under conditions in which the cells produce the polypeptide.

In one embodiment, cells which have taken up the nucleic acid are separated from cells which have not taken up the nucleic acids.

In another embodiment, the recombinant polypeptide is isolated from the cells.

It is preferred that storage-stable competent cells prepared according to the methods of the invention and stored at room temperature maintain a transformation efficiency of at least 10⁶ transformants/μg DNA for at least 2 months, and more preferably at least 3 months, 4 months, 5 months, 6 months or more.

In one embodiment, competent cells are dried under vacuum (preferably, under pressures of from 1000-3000 mtorr). In a further embodiment, cells are dried under vacuum for 2-24 hours at room temperature (e.g., from 15-30° C.). Still more preferably, cells are dried at 30° C. for 6-48 hours, and preferably for at least 8 hours. Longer drying times, e.g., 12 hours, 14 hours, 18 hours or 22 hours or more can also be used. In a further embodiment according to the invention, storage-stable competent cells are provided which maintain a transformation efficiency of at least 1×10⁵ transformants/μg DNA for greater than a month at room temperature.

In one embodiment, at least 5%, at least 10%, or at least 15% of the storage-stable competent cells are viable upon rehydration. In another embodiment, at least 20% of the cells are viable upon rehydration. In a further embodiment, at least 30% of the cells are viable upon rehydration, and preferably more.

The invention additionally provides kits comprising room temperature stable competent cells which can be shipped to a user without packaging in dry ice or with frozen packaging materials, eliminating costly overnight shipping expenses. In one embodiment, a kit according to the invention comprises a composition comprising a mixture of glass-forming matrix material and cells, wherein the Tg of the mixture is greater than 15° C., greater than room temperature, greater than 20° C., greater than 30° C., greater than 40° C., greater than 45° C., or greater than 50° C. or more. In a further embodiment of the invention, the kit comprises a sample of nucleic acids (e.g, such as lyophilized nucleic acids), and optionally, rehydration media, and instructions on how to rehydrate the cells and use them in transformation procedures. In a further embodiment, room temperature stable competent cells are packaged in a sealed pouch and optionally provided along with a desiccant, with instructions for reconstituting the cells for transformation. In a further embodiment of the invention, cells are provided along with a sample of supercoiled plasmid DNA, for example, to serve as a control to monitor the transformation efficiency of the competent cells.

### BRIEF DESCRIPTION OF DRAWINGS

FIG. 1 is a chart that shows the stability of room temperature competent cells grown in the presence of increasing amounts of NaCl.

FIG. 2 is a chart that shows the transformation efficiency (TE) and survival of room temperature competent XL10Gold (CamR) cells prepared with various desiccation media, competence medium, and growth medium.

FIG. 3 is a chart that shows the transformation efficiency (TE) of room temperature competent XL10Gold (Cam) cells prepared with various desiccation media, competence medium, and growth medium.

FIG. 4 is a graph showing the relative transformation efficiencies of chemicompetent B71 (sc19) cells that were dried in the presence of various concentrations of Sucrose, Trehalose, Sorbitol, Betaine, Inulin, or simply FSB and DMSO.

FIG. 5 is a chart showing the effect of using growth media containing fructose and proline/threonine on the transformation efficiency (TE) and survival of chemicompetent room temperature competent cells (RTCC).

FIG. 6 is a chart that presents a summary of the improvements made in room temperature chemicompetent cells as described herein.

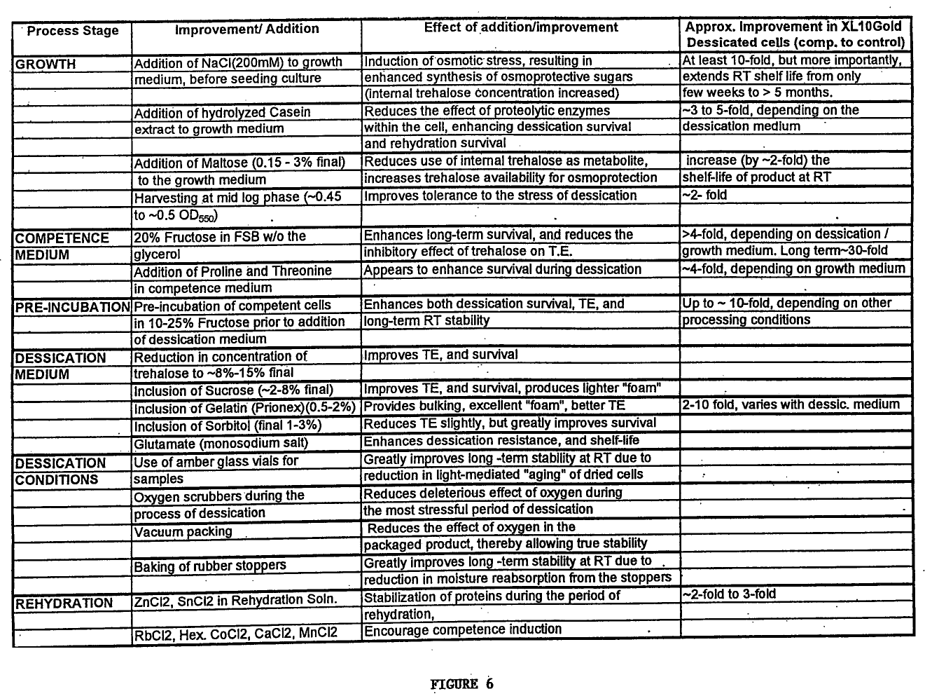

### DETAILED DESCRIPTION OF THE INVENTION

Room temperature stable competent cells provide vehicles for cloning and stably propagating nucleic acids of interest and for producing desired polypeptides. The competent cells according to the invention can be stored long-term (e.g., greater than a month) without the need for a −80° C. storage facility and can be shipped without the use of ice or other frozen packaging materials.

Definitions

In order to more clearly and concisely describe and point out the subject matter of the claimed invention, the following definitions are provided for specific terms which are used in the following written description and the appended claims.

As used herein, the term “hyperosmotic concentration of salt” refers to a salt concentration outside a cell, e.g., in a culture medium, that is at least 10% higher than the salt concentration inside the cell. Hyperosmotic concentrations of salt as used herein are preferably about 50 mM higher than isoosmotic, about 100 mM higher than isoosmotic, about 150 mM higher, about 175 mM higher, about 200 mM higher, about 225 mM higher, about 250 mM higher, about 275 mM higher, about 300 mM higher, about 325 mM higher or even about 350-400 mM higher, but are most preferably about 200 mM higher than isoosmotic.

As used herein, “stably stored” or “storage-stable” refer to cells which are able to withstand storage for extended periods of time (e.g., at least one month, or two, three, four, six, or twelve months or more) with a less than 95%, 90%, 80%, 70%, 60%, 50%, 40%, 30%, 20%, 15%, 10%, 5%, or 1% decrease in viability and which retain a transformation efficiency of at least 1×10⁵ transformants/μg DNA, and preferably at least 1×10⁶ transformants/μg DNA, 2×10⁶ transformants/μg DNA, 5×10⁶ transformants/μg DNA, 7.5×10⁶ transformants/μg DNA, or even 1×10⁷ transformants/μg DNA or even 5×10⁷ transformants/μg DNA or more.

As used herein, the term “competent cell” refers to a cell which has the ability to take up and replicate an exogenous nucleic acid, and preferably to produce viable clonal progeny comprising the exogenous nucleic acid.

As used herein, the term “defined solution” refers to a solution in which consists essentially of known components of defined chemical composition in known amounts. A “defined chemical composition” is a composition which can be expressed by a specific chemical formula, e.g., a salt or a carbohydrate, as opposed to a composition that cannot be expressed as a specific chemical formula, e.g., casein hydrolysate or yeast extract. A “defined solution”, as the term is used herein, excludes cell culture media, regardless of the presence or absence of components having undefined compositions.

As used herein, the term “non-reducing sugar” refers to a carbohydrate that does not reduce alkaline solutions of copper or silver. Non-reducing sugars do not participate in the Maillard reaction with proteins or amino acids. The reducing or non-reducing nature of a sugar is determined, among other ways, by Fehling's test, which monitors the reduction of Cu⁺⁺ to Cu⁺, with concomitant oxidation of the sugar. Non-limiting examples of non-reducing sugars include trehalose, sucrose, α-methyl glucoside, melezitose, raffinose, stachyrose and sugar alcohols, such as sorbitol. Functional equivalent forms of non-reducing sugars, such as methylated and chlorinated derivatives of fructose, glucose, maltose, or sorbose etc. can also be used, wherein functional equivalents refer to forms that are osmoprotective to the cells. In the present invention, mixtures of different non-reducing sugars can be used, as well as mixtures of both reducing and non-reducing sugars.

As used herein, the term “reducing sugar” refers to a carbohydrate that reduces an alkaline solution of copper or silver. Reducing sugars participate in the Maillard reaction with proteins and amino acids. Non-limiting examples include fructose, glucose (dextrose), maltose, lactose, glucopyranose, ribose and cellobiose. In the present invention mixtures of different reducing sugars can be used, as well as mixtures of both reducing and non-reducing sugars.

As used herein, the term “temperature above freezing” refers to a temperature greater than that at which at which a given liquid, e.g., a liquid cell suspension, becomes a solid. For pure water, a “temperature above freezing” refers to a temperature above 0° C. However, for an aqueous solution of a solute, e.g., a salt, carbohydrate, or protein, the freezing point will be lower than 0° C.; a “temperature above freezing” for such a solution can thus be below 0° C.

As used herein, the term “room temperature” refers to temperatures greater than 4° C., preferably from 15′-40° C., 15° C. to 30° C., and 15° C. to 24° C., and 16° C. to 21° C. Such temperatures will include, 14° C., 15° C., 16° C., 17° C., 18° C., 19° C., 20° C., and 21° C.

As used herein, the term “chemical agent” refers to a known chemical or combination of chemicals that, upon exposure to cells, makes those cells competent to take up exogenous nucleic acid.

As used herein, the term “dried cells” refers to cells retaining less than 10% residual moisture, preferably, under 5%, even more preferably, less than 3.5-4% residual moisture, most preferable about 2-3% residual moisture.

As used herein the term “casein hydrolysate” refers to a preparation of hydrolyzed casein, generally, although not necessarily, from bovine milk. A casein hydrolysate can be acid hydrolyzed or enzymatically (e.g., pancreatic digest, tryptic digest, etc.) hydrolyzed. Casein hydrolysate useful according to the invention is preferably an enzymatic hydrolysate. Casein hydrolysates are available from a number of sources, e.g., Sigma (St. Louis, Mo.).

As used herein, the term “transformation efficiency” refers to the number of transformed colonies formed with a given transformation reaction per unit mass of DNA added. Transformation efficiency is generally expressed in terms of transformed colonies per microgram of input DNA. Transformation efficiencies for re-constituted storage-stable competent cells of the invention are preferably at least 10⁵ colonies/μg.

As used herein, a “saccharide” refers to one or more of a disaccharide, trisaccharide, tetrasaccharide, oligosaccharide, polysaccharide and polymers of such saccharides. The term “oligosaccharide” refers to saccharides of from about 5 to about 10 sugar units having molecular weights, when unsubstituted, from about 650 to about 1300. The term “polysaccharide” refers to saccharides comprising greater than about 10 sugar units per molecule.

As used herein, a “derivative” refers to a compound with one or more substituents which still retains the function of the original compound or has improved function. For example, a disaccharide, oligosaccharide, or polysaccharide “derivative” refers to a disaccharide, oligosaccharide, or polysaccharide, respectively, comprising one or more atoms substituted by one or more other atoms, so long as the disaccharide, oligosaccharide, or polysaccharide has one or more, and preferably all, of the properties of being nonreducing or slowly reducing, non-crystallizing upon drying, forming a hydrate when water is absorbed, and comprising a Tg in the range of 10° C. to 80° C. and preferably, in the range of 30° C. to 60° C.

As defined herein, the term “exogenous DNA” refers to any of: plasmids, cosmids, DNA libraries, cDNA libraries, expression vectors, eukaryotic DNA, phage DNA, phagemid DNA, microbial DNA, single-stranded DNA, double-stranded DNA, supercoiled DNA, circular DNA, linear DNA, and the like.

As used herein, “a vector” is a DNA molecule which comprises an origin of replication and is capable of replicating extrachromasomally.

As defined herein, a “a selectable marker gene” is a gene encoding a marker that can be used to identify the presence of an exogenous DNA in a transformed cell (a cell, or progeny of a cell, which has been contacted with exogenous DNA and which has taken up the DNA). Selectable marker genes include, but are not limited to, drug resistance genes (e.g., antibiotic resistance genes), genes encoding detectable polypeptides (e.g., Green Fluorescent Protein), and genes encoding enzymes (e.g., which can be detected through the catalysis of their substrates), such as β-galactosidase, as well as unique sequences (e.g., producing signature restriction fragments) not found in the genome of the host cell being transformed.

As used herein, a “derivative” of a bacterial strain is a bacterium which comprises one or more mutations compared to a progenitor bacterial strain (e.g., the strain from which the derivative is “derived”) or one or more exogenous sequences compared to a progenitor strain. Mutations can be naturally occurring or induced through exposure to one or more mutagens and/or through the introduction exogenous DNA which recombines with the bacterial genome.

As used herein, a “Gram negative” bacterium is one which does not retain crystal violet or methylene blue in a standard Gram stain procedure. A “Gram positive” bacterium is one which retains crystal violet or methylene blue dye in a standard Gram stain procedure. The Gram staining procedure is well known to those skilled in the art.

As used herein, the term “Gram negative enteric bacteria” refers to Gram negative rod bacterial species that inhabit or are known to colonize the gut of mammals.

As used herein, the term “absolute vacuum” refers to a vacuum pressure less than 1 mTorr. Standard laboratory vacuum pumps are routinely capable of drawing a vacuum of 10⁻4 Torr or less, e.g., 10⁻6 Torr.

As used herein, the term “limiting the exposure to oxygen” means a set of conditions or a treatment that reduces the amount of free oxygen available to react with a given composition. By “limiting” or “reducing” in this context is meant at least a 10% reduction, and preferably at least 20%, 30%, 40%, 50%, 60%, 70%, 80%, 90% or more, relative to a sample that is not subject to such set of conditions or treatment. Exposure to oxygen can be limited in a number of ways, including, for example, de-gassing solutions under vacuum, flushing apparatus chambers with inert gas, and including oxygen scavengers or “scrubbers” in the desired location.

As used herein, the term “oxygen scavenger” or “oxygen scrubber” refers to a composition that tends to withdraw oxygen from the composition's environment, rendering the oxygen essentially incapable of reacting with surrounding materials. Oxygen scavengers include, for example, oxygen absorbing papers and sachets available from EMCO Packaging Systems (Kent, UK) and from Desiccare, Inc. (Pomona, Calif.).

As used herein, the term “limiting the exposure to moisture” means a set of conditions or a treatment that reduces the amount of water available to interact with a given composition. By “limiting” or “reducing” in this context is meant at least a 10% reduction, and preferably at least 20%, 30%, 40%, 50%, 60%, 70%, 80%, 90% or more, relative to a sample that is not subject to such set of conditions or treatment. Exposure to moisture can be limited in a number of ways, including, for example, storage in sealed containers, storage in the presence of a desiccant (e.g., silica gel), or pre-baking equipment or supplies to remove absorbed water or water vapor.

As used herein, the term “limiting the exposure to light” means a set of conditions that reduces the amount of light energy to which a given composition is exposed. By “limiting” or “reducing” in this context is meant at least a 10% reduction, and preferably at least 20%, 30%, 40%, 50%, 60%, 70%, 80%, 90% or more relative to a sample that is not subject to such set of conditions. Exposure to light energy is most readily limited by storing compositions in containers that transmit a reduced amount or no light energy. Examples include, but are not limited to storage in foil containers or storage in colored vials, particularly, for example, dark amber vials. A vial has “reduced transmittance of light” if it transmits less than 90% of the light energy exposed to the exterior of the vial to the inside of the vial, and preferably less than 80%, less than 70%, less than 60%, less than 50%, less than 40%, less than 30%, less than 20%, or even less than 10% of the ambient light.

Cells

A variety of cells, prokaryotic and eukaryotic (e.g., cells such as fungi, including yeast), can be rendered competent for transformation. In a preferred aspect, the cells are bacterial cells and include, but are not limited to, Gram negative and Gram positive bacterial cells, such as Eschericia sp. (e.g., E. coli), Klebsiella sp., Salmonella sp., Bacillus sp., Streptomyces sp., Streptococcus sp., Shigella sp., Staphylococcus sp., and Pseudomonas sp. Preferred species include Gram negative enteric bacteria, generally Gram negative bacilli, including, for example, Eschericia sp., Salmonella sp. (e.g., S. enteritidis, S. typhimurium), Vibrio sp. (e.g., V. cholerae, V. parahemolyticus), Shigella sp. (e.g., S. dysenteriae, S. sonnei), Campylobacter sp. (e.g., C. jejuni) and Yersinia sp. (e.g., Y. pestis, Y. enterocolitica).

In a preferred embodiment of the invention, E. coli strains are rendered competent for transformation by exogenous nucleic acids. Suitable E. coli strains include, but are not limited to, BB4, C600, DH5, DH5a, DH5a-E, DH5aMCR, DH5a5′IQ, DH5a5′, DH10, DH10B, DH10b/p3, DH10BAC, HB101, RR1, JV30, DH11S, DM1, LE392, SCS1, SCS110, Stab2, DH12S, MC1061, NM514, NM522, NM554, P2392, SURE®, SURE 2, XL1-Blue, XL1-Blue MRF, XL1-BlueMR, XL2-Blue, JM101, JM109, JM110/SCS110, NM522, TOPP strains, ABLE®, XLI-Red, BL21, TK B1, XL10-Gold® Cells, Restriction-Minus Competent Cells™, TK Cells, ABLE® strain, XlmutS strains, SCS110, AG1, TG1, SOLR™, XLOLR strain, Y1088, Y1089r, Y1090r-strains, WM100, and derivatives thereof. Information relating to the genotypes of these strains are known in the art and can be found, for example, at www.stratagene.com.

Methods of Making Competent Cells According to the Invention

A. Growth of Cells

Cells are first grown in a medium that supports cell proliferation. Cell growth medium encompassed within the scope of the invention includes, but is not limited to: Luria Broth; Psi broth (e.g., 5 grams bacto yeast extract, 20 grams Bacto tryptone, 5 grams of magnesium sulfate, per liter); SOB medium (e.g., 0.5% yeast extract, 2% tryptone, 10 mM NaCl, 2.5 mM KCl₂, 10 mM MgCl₂, 10 mM MgSO₄); SOC medium (e.g., 2% tryptone, 5% yeast extract, 2.5 mM KCl, 10 mM NaCl, 10 mM MgCl₂, 20 mM glucose); Terrific Broth (“TB”) (e.g., 12 grams of tryptone, 24 grams of yeast extract, 4 ml of glycerol 2.3 grams of KH₂PO₄, 12.5 grams of K₂HPO₄, per liter); TY medium (8 grams of tryptone, 5 grams of NaCl, 5 grams of yeast extract, per liter, adjusted to pH 7.2-7.4 with NaOH), and other media used to support the growth of cells, such as bacteria. In one embodiment, the cell growth medium used is supplemented to comprise additional growth-promoting agents (e.g., vitamins, sugars, ions, and the like). It should be obvious to those of skill in the art that a variety of media can be used, and that such media are encompassed within the scope of the invention.

For some strains, the inclusion of casein hydrolysate (enzyme-hydrolyzed) in the growth medium at a concentration in the range of 11-15 g/l enhances long-term survival up to 2 fold. It is preferred that the enzymatic casein hydrolysate be included at a concentration of at least 11-12 g/l.

An increase of 10 to 100-fold in competent cell survival after drying can be achieved by growing the cells in the appropriate growth medium with a salt concentration increased above the standard amount. The actual increase in cell survival for cells cultured in the presence of increased salt concentration is dependent upon other factors influencing the bacterial physiological response to stress, such as sugars in the competence medium, composition of pre-desiccation medium, composition of the desiccation medium, and drying parameters described elsewhere herein. Generally, useful hyperosmotic concentrations of NaCl are 100 mM to 350 mM above the isoosmotic salt concentration, preferably about 200 mM to 250 mM above iso-osmotic. The mild osmotic shock induced by growth in the presence of increased salt appears to enhance the production of osmoprotective substances by the cells, thereby increasing survival in the subsequent drying step.

An additional increase in desiccation tolerance of approximately 2-5 fold can be achieved by supplementing the growth medium with low concentrations of maltose, e.g., 0.1% to 0.3% (weight to volume), preferably about 0.2% to 0.3%.

It has long been known that the growth stage of the culture is an important determinant of cell competence. However, the growth stage of the culture also appears to influence the survival of the competent cells during and after drying. Our fairly extensive studies in optimizing the OD or growth phase for competent cells later subjected to drying has clearly shown that there exists a direct relationship between the stage or phase of growth and the desiccation tolerance. Typically an overnight culture of a bacterial cell strain, or a frozen competent cell stock of the strain is inoculated into NaCl-fortified growth medium, optionally including maltose and casein hydrolysate, and grown at 37° C. in a shaker or fermenter to a final OD₅₅₀ of 0.48 to 0.5. Cells in the late log or stationary phase of growth are generally more resistant to the stress of desiccation, but also have lower competence (that is, while it is possible to generate chemically competent storage-stable cells from cultures harvested at OD₅₅₀ as high as 0.8 to 0.85, the competence achievable for such cells will be lower than for cells harvested at earlier growth stages). An optimal balance between desiccation tolerance, cell density, and chemical competence was arrived at when cells were harvested when cultures reached an OD₅₅₀ of ˜0.45 to 0.5. If necessary, media can be reinoculated with cells which have reached mid to late stationary phase to reinitiate log phase growth.

Incubation temperatures for growing cells can vary from 10° C. to 42° C., but preferably ranges from 20° C. to 40° C. In one embodiment according to the invention, cells are grown with shaking to promote aeration, (e.g., at 100 to 500 revolutions per minute (rpm)).

B. Cell Harvesting and Treatment to Induce Competence

Upon reaching the optimal OD, cells are quickly chilled on ice-water baths for 30 minutes, and then harvested. Cells at a desired stage of growth are harvested, for example, by centrifugation, filtering, allowing cells to settle, by size exclusion chromatography, etc. Gentle harvesting by centrifugation is preferred. For example, cells are centrifuged in pre-chilled 50 ml round-bottomed tubes (e.g., Nalgene tubes), at 1600 rpm, for 7 minutes, at 4° C. in a Beckman J-6B rotor and centrifuge). Harvested cells are then resuspended in a suitable buffer for competence induction.

A number of different competence induction protocols are known to those skilled in the art. Suitable competence-inducing buffers (also sometimes referred to as “transformation buffers”) include, but are not limited to, 50 mM CaCl₂, 10 mM Tris/HCL (Sambrook, et al., Molecular Cloning: a Laboratory Manual, 2nd Edition, eds. Sambrook, et al., Cold Spring Harbor Laboratory Press, (1989)); TB buffer (e.g., 10 mM PIPES, 15 mM CaCl₂, 250 mM KCl) (Inoue, et al, Gene 96: 23-28 (1990)); 2×TSS (LB broth with 10% PEG (MW3350-8000), 5% DMSO, and 20-50 mM Mg²⁺ (MgSO₄ or MgCl₂) at a final pH of 6.5) (Chung, et al., PNAS 86: 2172-2175, (1989)); FSB buffer (e.g., 10 mM potassium acetate, 100 mM KCl, 44 mM MnCl₂, 10 mM CaCl₂, 3 mM HACoCl₃, 10% redistilled glycerol) (Hanahan, D., In: DNA Cloning (D. M. Glover, ed) IRL Press, Washington, D.C., pp. 109-135); and CCMB80 buffer (10 mM potassium acetate pH 7.0, 80 mM CaCl₂, 20 mM MnCl₂, 10 mM MgCl₂, 10% glycerol, adjusted to pH 6.4 with 0.1N HCl) (Hanahan, et al., Methods in Enzymology 204: 63-113 (1991)). The entirety of these references and all others cited herein are incorporated herein by reference.

Where competence-induction buffers call for glycerol, it should be omitted when used in the methods of the invention. The inclusion of glycerol tends to reduce the Tg of the final dried cell product, thereby reducing storage stability.

Preferably, cells to be made competent are resuspended in transformation buffer which has been pre-cooled to 4° C. (e.g., by chilling on ice). Generally, cells are exposed to competence-inducing buffer for at least 2-60 minutes.

Methods of making competent cells can be selected to suit a user's needs. For example, when transforming cells with supercoiled plasmid DNA, generally any method known in the art will provide an acceptable number of transformants (e.g., 1 per agar plate). However, for clones comprising unstable or less stable sequences (e.g., LTR sequences and inverted repeats), it may be desirable to alter growth conditions to enhance the stability of the cells, i.e., such as by growing cells at lower temperatures (25° C. to 30° C.) in rich medium (e.g., TB broth) and by ensuring that growth does not continue beyond OD₅₅₀ 0.45 to 0.5. Alternatively, or additionally, cells whose genotypes minimize rearrangements of unstable sequences can be used (e.g., such as STBL strains). Where limiting amounts of cloned sequences are to be introduced into a cell, transformation buffers can be additionally supplemented by agents for enhancing transformation efficiency, including, but not limited to, hexamine cobalt chloride, sodium succinate, RbCl, and the like (see, as discussed in U.S. Pat. No. 4,981,797, the entirety of which is incorporated by reference herein).

Additional methods of generating competent cells are described in: Kushner, In: Genetic Engineering: Proceedings of the International Symposium on Genetic Engineering, Elsevier, Amsterdam, pp. 17-23 (1978); Norgard, et al., Gene 3: 279-292 (1978); Jessee, et al., U.S. Pat. No. 4,981,797, the entireties of which are incorporated by reference herein, and on the World Wide Web at www.protocol-online.net/molbio/DNA/transformation.html. Generally, competence is higher in Gram positive bacteria than in Gram negatives.

The desiccation tolerance and chemically induced competence of cells can be enhanced by several treatments performed either prior to or concurrently with the induction of competence. It is known in the art that cells can be protected during drying by including glass-forming matrix materials (see below), particularly non-reducing saccharides, in the drying medium. Although glass-forming saccharides perform well in maintaining cell viability during desiccation, high concentrations of certain non-reducing sugars also tend to inhibit the chemically-induced competence, i.e., the transformation efficiency, of the re-constituted bacteria. Thus, high viability is often accomplished at the expense of reduced competence.

One means of counterbalancing this adverse effect of the non-reducing sugar on competence is to treat competent cells, which have already been salt-stressed to induce the accumulation of internal osmoprotective sugars, with an appropriate reducing or non-reducing sugar. According to the invention, treatment with such sugars is beneficial if it is performed before, concurrently with, or after (i.e., immediately before desiccation) the induction of competence. For example, cells may be processed on ice either before, during, or after competence induction, in the presence of appropriate reducing sugars, such as fructose, or non-reducing sugars/sugar alcohols such as sorbitol, α-methyl glucopyranoside, etc., at a final concentration of 10% to 25% (w/v). However treatment with sugars during or after competence induction is preferred. Pre-treatment with these advantageous sugars can be performed by harvesting salt-stressed cells at the optimal OD₅₅₀, inducing competence, then resuspending the cells in a solution of 10%-25% reducing sugar (e.g., fructose) or the non-reducing sugar, e.g. sorbitol, α-methyl glucopyranoside, etc. and incubating for 10 minutes on ice. For pre-treatments, cells are resuspended in small volumes of the sugar solution, e.g., 50 μl of sugar solution per 40 ml of original culture volume.

In one aspect of the invention, the reducing or non-reducing sugar solution comprises a mixture of the preferred sugars at a final concentration of 20% [in the competence medium, or after competence induction] for example 10% sorbitol and 10% α-methyl glucopyranoside, or 10% fructose and 10% sorbitol, or, single sugars in a final concentration of ˜20%, e.g. 20% fructose, or 20% sorbitol, etc.

In another aspect of the invention, the cells are made competent with the appropriate competence medium, gently harvested, then treated with a very small volume of 20% solution of sugar or sugar mixtures immediately prior to addition of the desiccation/preservation medium. This treatment can be done at very low temperatures (0°-4° C.) so that the cells retain their competency, while being enriched for (absorbing) the osmospective sugars.

For sugar treatment concurrent with competence induction, the reducing sugar, e.g., fructose, or the non-reducing sorbitol, is simply included in the competence-inducing buffer (e.g., FSB without glycerol). For this method, the reducing sugar is included in the competence-inducing buffer at a concentration of 7%-30%, preferably about 20%. Aside from the addition of the reducing sugar to the competence-induction buffer, all other aspects of competence induction remain the same. For example, the cell suspension is incubated on ice for 18 minutes (time can be varied) and then gently centrifuged and resuspended in desiccation medium. As an added benefit, treatment with reducing sugar improves not only the competence of the cells after re-constitution, but also the long-term survival of the dried cells at temperatures above −80° C. (e.g., room temperature). The increase in transformation efficiency with reducing sugar (e.g., fructose) or the non-reducing sugar/sugar alcohol (e.g. sorbitol) treatment, either in the competence inducing buffer or immediately prior to addition of desiccation medium, is generally about 2-10-fold.

Long-term storage survival at temperatures above −80° C. (e.g., room temperature) can also be enhanced by approximately 4-fold by including amino acids with stress-relieving properties, such as proline and threonine, in the competence-inducing buffer. When used, proline and/or threonine should be included in the buffer at a final concentration of approximately 2 mg/ml to 4 mg/ml.

Generating Room Temperature Stable Competent Cells

In order to prepare room-temperature-competent cells, competent cells prepared by any of the methods described above, or by any methods known in the art, are contacted with a solution comprising a water soluble glass-forming matrix material. In one embodiment, the glass-forming matrix material is hydrophilic and comprises a glass transition temperature (“Tg”) from 10° C. to 80° C. upon drying, and preferably comprises a Tg of at least 40° C., and still more preferably, comprises a Tg of at least 45° C., at least 50° C., or at least 60° C.

Suitable glass-forming matrix materials include carbohydrates, such as non-reducing sugars, which minimize oxidative damage to the cells. In one embodiment, the matrix material is a saccharide selected from the group consisting of trehalose, sucrose, melzitose, raffinose, maltitol, sorbose, lactitol, dextrose, sugar alcohols (e.g., sorbitol, galactitol, mannitol, xylitol, erythritol, threitol, sorbitol glycerol, polyglycerols, such as diglycerol and triglycerol, and the like), sugar ethers (e.g., sorbitan and polyvinyl alcohols), sugar acids (e.g., L-gluconate), and derivatives and combinations thereof. Polysaccharides such as amylose, ficoll™ (see, U.S. Pat. No. 3,300,474), dextrin, starch, dextran, and polydextrose also can be used.

The useful concentration or concentrations of glass forming matrix material(s) can vary from about 4% to 25%, either as a single sugar or as a mixture of two or more sugars or sugar alcohols. In one embodiment, the competent cells are contacted with a 20% carbohydrate solution, such as 20% trehalose, 20% sucrose, 20% melezitose, or 20% raffinose. In another embodiment, the cells are exposed to a solution which comprises 10% or less of two different carbohydrate solutions (e.g., 10% trehalose and 10% melzitose; 10% raffinose and 10% trehalose; 10% raffinose and 10% melzitose; 10% trehalose and 10% sucrose; 10% raffinose and 10% sucrose; 10% melzitose and 10% sucrose, etc.). In one aspect, the matrix material is supplemented with a sugar alcohol, such as sorbitol, preferably at a concentration of 2.5% w/v.

In a preferred embodiment, a saccharide is used which does not crystallize upon drying and which comprises a Tg in the range of 10° C. to 80° C., preferably in the range of 30-80° C., and more preferably in the range of 60-80° C. In one embodiment, the glass forming matrix material is a non-reducing carbohydrate selected from the group consisting of disaccharides, trisaccharides, tetrasaccharides, oligosaccharides, polysaccharides, sugar alcohols, sugar ethers, sugar acids, and derivatives and combinations thereof.

Preferred saccharides include, but are not limited to, trehalose, raffinose, melezitose, sucrose, maltitol, derivatives thereof, and combinations thereof. In one embodiment, a glass-forming saccharide is selected which hydrolyzes into a reducing sugar at a slow rate (e.g., such as trehalose). In another embodiment, a saccharide is selected which forms a hydrate when water is absorbed, thereby maintaining a high Tg (>15° C., preferably greater than 40° C., more preferably, greater than 50° C., and still more preferably, greater than 60° C.) upon drying.

Other glass-forming matrix materials are known and are encompassed within the scope of the invention. These include, but are not limited to, polyols. In addition to sugar polyols, polyols such as propylene glycol and polyethylene glycol, also can be used, as can polymers such as polyvinylpyrolidone, polyacrylamide, polyethyleneimine, albumin, and the like.

Competent cells which have been previously frozen can be used, as well as competent cells which are freshly made (e.g., less than 2 hours old) and have been stored at −20° C. to 4° C. However, preferably, the cells are not frozen immediately prior to drying (i.e., at least 1 minute prior to drying). In one embodiment, the competent cells are collected by centrifugation (e.g., to substantially remove transformation buffer), and resuspended in a solution comprising the glass-forming matrix material.

The formulation of the desiccation medium is critical not just to the process of drying and generating a light and easily re-hydratable foam or dried product, but also heavily influences the eventual stability of the dried competent cells. The stability of the dried product involves not only the desiccation tolerance during desiccation, but also tolerance to oxidation, and maintenance of a “buffering” and “bulking” matrix which will minimize enzymatic reactions within the cells. In essence, the optimal desiccation medium should have a mixture of components which will allow for or promote the following:

1. Accelerated “fluffing” or foaming of the cell suspension, and accelerated drying of the fluffy layers of foaming product. A quick drying ensures that the drying is efficient, and that there is reduced exposure of the cells to the aqueous sugars in the desiccation medium. In order for this to happen, one has to ensure that the desiccation matrix is not too heavy. Gelatin is one agent that satisfies these requirements, particularly modified hydroyzed porcine gelatin (e.g., Prionex, Type A, Sigma). For example, Prionex gelatin, a liquid suspension containing about 10% solids, is diluted 1:10 in desiccation medium, which is then added to the cells, for a final concentration of gelatin in the medium of about 0.5% (0.5% to 2.5% final concentration is preferred). In combination with a mixture of trehalose and sucrose, both of which are osmoprotective, plus glutamic acid (monosodium salt; about 1-4 mg/ml of desiccation medium), the gelatin ensures ideal foaming under appropriate vacuum pressures, and good bulking. Sucrose, which foams more readily than trehalose, also adds to this property.

2. Tg values close to or higher than ambient/room temperatures: An appropriate combination of the above ingredients provides a dried product Tg that is well above ambient temperatures, ensuring longer product stability. Inclusion of trehalose (final concentration of approximately 7% to 8%) improves the Tg of the product, while ensuring reduced ill-effects on transformation efficiency. The total concentration of all sugars in the desiccation medium should preferably not exceed about 25% w/vol. Methods of measuring Tg are described herein below.

3. Bulking: Agents which improve the bulking of dried products enhance the shelf-life largely because they reduce the contact between individual dried biomolecules (in this case, the competent cells). Hence, deleterious by-products from dead or dying cells will have a lesser effect on cells which have survived the desiccation process, and this in turn helps in extending the survival at room temperature. Gelatin, in its many forms and sources, has excellent bulking properties. A preferred form is hydrolyzed modified porcine gelatin (e.g., Prionex Gelatin, Sigma) which has both excellent bulking and foaming properties. Preferred concentrations of gelatin in the desiccation medium are described herein above.

4. Resistance to easy water absorption: While the dried cells need to be easily hydratable in the end-user's hands, a certain amount of insensitivity to moisture is very valuable. The dried product may absorb water at different stages during its storage. Trehalose has the advantageous property of reducing the distribution of absorbed water in the dried product (by segregating the water), thereby ensuring that the majority of the dried product stays dry and viable even with the absorption of some moisture.

Cells suspended in the selected desiccation medium are dried under vacuum. Traditional approaches freeze-dried the cells, which literally involved freezing the cell suspension solid (often to −80° C. or below) and then exposing the frozen mass to high vacuum in order to remove water by sublimation. However, this traditional approach yields poor viability with the relatively fragile Gram-negative cells, and particularly those that have been rendered even more fragile by treatment to induce chemical competence. The invention provides an alternative process optimized to yield greatly improved cell viability. In this process, the competent cells are suspended in the desiccation medium and exposed directly to a regulated vacuum source, i.e., without freezing. This results in a “foaming” action as water rapidly evaporates in the carbohydrate-rich cell suspension. The foaming action aids in coating cells in the sugar glass; the better the foaming action, the better the protection of the cells during the desiccation process.

The concentration of cells in the desiccation medium influences the survival and storage-stability of the cells. An excess of cells in the desiccation medium can lead to a poor foam, resulting in insufficient coating of the cells in the carbohydrate glass, which reduces storage stability. Cells should generally be present at a concentration of about 10⁹ to 10¹¹ cells per ml of desiccation medium

Sugar concentration in the desiccation medium is also a critical parameter. While sugar or sugars are necessary for protecting the cells, an excess could lead to possibly excessive sugar absorption, with a consequent reduction in transformation efficiency. This is because the cells are exposed to the sugars in the aqueous environment twice immediately before desiccation, and immediately upon re-hydration. Extremely high sugar concentrations can also reduce the viability of the dried cells. Sugar concentration in the desiccation medium should be from about 4% to about 25%, for example 4%, 6%, 8%, 10%, 12%, 14%, 16%, 18%, 20%, 22%, 24% or 25%. A concentration of about 10% is preferred. The concentration of any one sugar will depend upon whether a single sugar or a mixture of sugars is used.

Aliquot size and the geometry of the desiccation vessel (tube, vial, etc.) are also important parameters that influence the drying efficiency and kinetics. Flat bottomed tubes are generally favored, in order to provide increased surface area relative to conical vials and tubes. Aliquot sizes that are too small tend to result in a “donut” shaped dried product that sometimes may not dry efficiently due to movement of the sample to the edges of the vial. At the other extreme, large volumes lead to poor foaming and drying because water cannot effectively escape from multiple layers of foaming product, trapping water vapor between the layers. The optimal volume of cell suspension will necessarily change depending upon the size of the tube used. However, as a guide, for a 13 mm diameter tube, the optimal aliquot size ranges from about 75 μl to about 200 μl.

Drying can be performed using standard drying apparatuses known in the art as lyophilizers, sublimators, Speedvacs™, and the like. Freeze-drying apparatuses can be modified for use in the process (e.g., by not drying in the presence of dry ice, or by setting a temperature control to a temperature above freezing, i.e., such as room temperature or above). The cells themselves are never freeze-dried. In one aspect of the invention, 4 liters of cells are aliquoted into vials at volumes of 150 μl per vial; using a sublimator capable of accommodating 4800 vials.

As water is removed from the competent cells during the drying process, the glass-forming matrix material forms an increasingly glassy amorphous matrix which surrounds the cells. This glassy matrix is stable at temperatures below the Tg; however, at temperatures above the Tg, the glassy matrix loses its structure and becomes more fluid, assuming a syrup-like consistency. Thus, any cells which are stored at temperatures above the Tg of the glass matrix-cell mixture will cease to be immobilized as the glass matrix becomes fluid and will be susceptible to degradation. Because the temperature stability of the glass matrix reflects the storage stable temperature of the cells, in one embodiment, drying conditions are selected which generate a glassy matrix-cell mixture having a Tg which is higher than or equal to the desired storage temperature of the cells, e.g., at least room temperature, preferably greater than 40° C., still more preferably greater than 45° C., greater than 50° C., and most preferably, greater than 60° C.

In one embodiment, optimal temperature and drying times are identified by drying cells, measuring the Tg of the glassy matrix-cell mixture as the drying process proceeds, and subsequently standardizing the drying procedure to achieve a desired Tg (i.e., a Tg at least as high as a desired storage and/or shipping temperature).

The Tg of the glass matrix-cell mixture can be measured using means standard in the art, such as by differential scanning calorimetry, dynamic thermal analysis (DTA), dynamic mechanical thermal analysis (DMTA), dynamic mechanical analysis (DMA), low field NMR, and the like. In one embodiment, the Tg of the matrix is determined at different time points during the drying process, to determine whether additional drying is required. It should be obvious to those of skill in the art that while different methods of measuring Tg's may yield slightly different results, whether the cell-glass matrix mixture has reached a desired Tg also can be verified experimentally, e.g., by determining whether the composition remains in a glassy form at a selected temperature, such as room temperature or higher. Thus, a cell having a Tg of a given temperature would remain in its glassy form (i.e., not liquefy) at temperatures of the given temperature and below.

In one aspect of the invention, the Tg of the matrix-cell mixture is increased by adding a glass-enhancing agent, e.g., such as a zwitterion comprising polar or apolar radicals, such as amino carboxylic acids, and their salts (see, EP-913178, the entirety of which is incorporated herein by reference), zinc ions or other metal ions (see, e.g., U.S. Pat. No. 4,806,343, the entirety of which is incorporated herein by reference), and borate. In another aspect, the glass-matrix material comprises a saccharide which is already hydrated (see, e.g., U.S. Pat. No. 6,071,428, the entirety of which is incorporated by reference herein) prior to being contacted with the competent cells to increase the Tg of the glass-matrix: cell mixture.

While drying under immediate vacuum without any stepwise adjustment of vacuum or temperature may lead to faster drying and some viable cells, a more gradual decrease in pressure appears to be better tolerated by the competent cells. One example of pressure conditions useful according to the invention is as follows: initially, a vacuum of approximately 2000 mTorr-1500 mTorr is applied for 30 minutes, followed by increasing the vacuum to approximately 1200 mTorr-1000 mTorr. This lower pressure can be maintained until the cells are dry, or, alternatively, can be further reduced in a step-wise fashion until the cells are dry.

The “dryness” of the cells is indicated in terms of residual moisture content. “Residual moisture content” refers to “bound” moisture that remains associated with the dried cells after primary drying. “Primary drying” refers to the drying of cells to constant weight under a first set of drying conditions. Primary drying results in a dried cell product that still has bound residual moisture that can be driven off by secondary drying, which is performed at higher temperature and, optionally, higher vacuum than primary drying. The amount of residual moisture can be evaluated by any method known to those skilled in the art, which include, but are not limited to, i) Karl Fischer Thermal method, ii) Thermogravimetry/Mass Spectometry (TG/MS), iii) Moisture evolution method and Vapor pressure moisture method, iv) gas chromatography, v) Near infared reflectance (NIR) spectroscopy, vi) Gravimetric (loss-on-drying) method, vii) Differential scanning calorimetry (DSC), and viii) Thermally stimulated polarization current (TSPC).

Competent cell preparations are “dry” according to the invention, when their residual moisture content is 10% or less by weight, preferably under 5%, even more preferably, less than 3-4% residual moisture, most preferably 2-3% residual moisture. Secondary drying at higher temperature and elevated temperatures can further reduce moisture content and enhance the long-term shelf-life of the dried cell product. Secondary drying conditions useful according to the invention are, for example, approximately 40-45° C. under absolute vacuum. Pressure can be optimized in consideration of the surface area of cells being dried, e.g., to take into account the type of container in which the cells are being dried.

Cells are dried according to the invention at a temperature above freezing, e.g., at room temperature. In a preferred embodiment, cells are dried at a temperature within the range of from 15° C.-40° C. In a more preferred embodiment, cells are dried at 30° C. According to the invention, small aliquots (50 ul, 100 ul, 200 ul, up to 500 ul) of competent cells that have been treated with osmoprotective sugars, are dried in the presence of non-reducing sugar(s) containing stabilizing additives such as sugar alcohol, namely sorbitol, gelatin, and glutamate, at temperatures above freezing, such that storage-stable competent cells are generated.

Drying times can be varied to achieve an optimal Tg. In one embodiment, the drying time ranges from 2-48 hours. In a preferred embodiment, the drying time ranges from 6-24 hours. More preferably, the drying time is at least 8 hours. In one embodiment of the invention, cells are dried at 30° C. from 18-24 hours, such that a drying run can be easily set up overnight while cells are prepared (i.e., rendered competent) during the day.

One example of temperature and pressure conditions useful according to the invention is as follows: Starting at 30° C., a vacuum of approximately 2000 mTorr-1500 mTorr is applied for 30 minutes, followed by increasing the vacuum to approximately 1200 mTorr-1000 mTorr. This lower pressure can be maintained until the cells are dry, or, alternatively, can be further reduced in a step-wise fashion until the cells are dry. Alternatively, vacuum drying of chemically competent cells can be performed at vacuum pressures starting as high as 25 Torr to as low as 10 mTorr. Preferably, the initial vacuum pressure is between about 1000 and 3000 mTorrs, and is maintained for several hours. One preferred method is to begin drying under a higher pressure of between 2000 and 2500 mTorrs, sufficient to produce foaming, but without losing sample as a result of sudden foaming. This pressure is maintained for about 10-30 minutes. Pressure is then reduced to between about 1500 to 1000 mTorrs, and maintained for several hours, up to 20-22 hours, or overnight. One to four hours prior to the end of the vacuum incubation time, the pressure is further reduced to between 8 and 200 mTorrs. Samples are subsequently vacuum stopped. Secondary drying can then be performed, for example, at approximately 40-45° C. under absolute vacuum. After secondary drying, vials are then stoppered under absolute vacuum or backflushed with Argon or Nitrogen before stoppering.

After drying is completed and a satisfactory Tg is obtained, dried cells are stored in sterile containers at room temperature until use. Where cells are dried in bulk, dried cells can be milled into powder and dispensed into individual containers suitable for use. In one embodiment according to the invention, cells are packaged in a form suitable for shipping, for example, by storing the cells in sealed pouches in the presence of desiccant.

The effectiveness of changes in the drying parameters, or, for that matter, any changes introduced into the growth, competence induction and/or drying processes described herein, can be measured by plating cells which have been treated under a given set of conditions, counting the number of colonies formed, and comparing these numbers to the numbers of colonies formed from plated cells which were treated under conditions with known results.

Post-Drying Measures to Increase Stability

Additional steps that can be taken to increase the shelf-life of the dried competent cells include:

1) Storage in photo-insensitive amber vials. Following desiccation, cells are very sensitive to factors that normally would not have caused stress under normal aqueous conditions. Among these factors is exposure to light. Thus, storage of the cells in amber vials can reduce this source of stress and increase shelf-life. Similarly, storage of the amber vials in a sealed foil pouch will also aid in reduction of light exposure.

2) Reduction in the exposure of dried/drying cells to oxygen. The exposure of the dried cell product to oxygen should be limited to avoid oxidation where possible. This can be done during and/or after drying. Means of limiting oxygen exposure include, for example, flushing the lyophilizer chamber with argon, nitrogen or other inert gas prior to lyophilization. Another means involves de-gassing the desiccation medium under vacuum prior to its addition to the cell pellet. Alternatively, oxygen scrubbers can be used during the process of drying, or placed in the vial or in its cap. Again, storage of stoppered vials in sealed foil pouches will aid in reducing exposure to oxygen, especially if oxygen scavengers are provided in the pouch.

3) Pre-baking of stoppers to reduce moisture. Rubber stoppers or those made of other materials used to cap vials of dried cells can contain a considerable amount of absorbed moisture. This moisture can be removed by baking the stoppers at, for example, about 116° C. for 4 to 5 hours. Storage of the vials in sealed foil pouches will also help to maintain the moisture-free mature of the stoppers and the vial contents, especially if a desiccant is also placed in the pouch.

How to Use Room Temperature Competent Cells According to the Invention:

Cell Transformation

Dried cells can be rehydrated for use in subsequent transformation procedures. In one embodiment, the dried competent cells are resuspended in an appropriate amount of water which does not lyse the cells; i.e., generally, at least a volume of water equal to the volume of stored competent cells. Preferably, cells may be further diluted in an appropriate buffer (e.g., transformation buffer) or cell growth media. In one embodiment, cells are rehydrated, collected (e.g., by centrifugation), and washed at least one time in a transformation medium or cell growth medium, to remove or substantially dilute, residual glass matrix forming material (e.g., to 5% w/v or less). Still more preferably, cells are immediately resuspended in an equal volume of chilled transformation buffer (e.g., such as FSB or FSB containing 2.5% to 5% DMSO, or CaCl₂, BaCl₂, SnCl₂, ZnCl₂, etc.), or Tfb1 broth (potassium acetate 30 mM, ribidium chloride 100 mM, calcium chloride 10 mM, manganese chloride 50 mM, final pH=5.8%, or Tfb II broth (MOPS 10 mM, CaCl₂ 75 mM, rubidium chloride 10 mM, 3% fructose, final pH 6.5). Transformation buffer medium is rehydration buffer or cell growth medium.

In one embodiment, rehydrated room temperature stable competent cells according to the invention are used in transformation procedures by contacting the cells with nucleic acids, preferably comprising a selectable marker gene (e.g., a gene encoding resistance to an antibiotic or expressing a detectable polypeptide, or enzyme which can catalyze a detectable reaction, such as β-galactosidase), and plating the cells on a plate containing a selection medium (e.g., an antibiotic or substrate for the enzyme).

Nucleic acids encompassed within the scope of the invention, include, but are not limited to, nucleic acid sequences that encode functional or non-functional peptide, polypeptides, proteins and fragments of those sequences, as well as nucleic acids which comprise non-coding sequences (e.g., regulatory sequences, such as promoters or enhancers). The nucleic acids may be natural (e.g., isolated from cells) or synthetic nucleic acids (e.g., obtained by PCR or mutagenesis of isolated nucleic acids, or chemically synthesized). The nucleic acids can be circular, linear, or supercoiled. Although not limited to particular sizes, in some embodiments, the nucleic acids used to transform the cells according to the invention range from 1.0 kb to 300 kb.

In one embodiment, competent cells which have been contacted with nucleic acids are incubated for 2 minutes to 2 hours at 4° C.-30° C. For chemically competent cells, preferably, a volume of cells rehydrated in transformation buffer are transferred to pre-chilled tubes and chilled on ice (e.g., are at 4° C.) for ten minutes, and incubated in the presence of exogenous DNA on ice for an additional 20 minutes. Contacted cells are plated onto agar plates comprising a suitable selection media, either directly, or after dilution in a cell growth medium (which can also be further incubated to promote cell growth). In one embodiment of the invention, cells are heat shocked at 20-42° C. for 30 seconds to 2 minutes, prior to plating. Preferably, cells are heat shocked at 42° C. for 60 seconds, transferred to ice for 2 minutes, and diluted in culture medium (e.g., generally, a 10:1 dilution). Cells are preferably incubated with aeration (e.g., shaking) for 1 to 10 hours prior to plating (preferably 1-2 hours) and then plated onto a solidified culture medium comprising the appropriate selection medium (e.g., antibiotics or a substrate if the exogenous DNA expresses an enzyme capable of catalyzing a substrate).

Transformation efficiencies of the storage stable cells generated according to the method range from at least 10⁵ to 10⁸ transformants/μg DNA, and preferably, at least from 10⁶ to 10⁸ transformants/μg DNA, while the viability of the cells is at least 5%, and preferably, at least 10-15% of the viability of cells prior to drying. In one embodiment, the viability of the cells is at least 20%, or at least 30%, of the viability of cells prior to drying.

Producing Recombinant Polypeptides Using Storage Stable Competent Cells

In a further embodiment, the invention provides a method of producing recombinant polypeptides (e.g., polypeptides expressed by the exogenous nucleic acids which have been used to transform the cells). In this embodiment, competent cells which have been transformed with a nucleic acid encoding a protein of interest are grown in a cell growth medium under conditions in which the cell will express the polypeptide (e.g., the polypeptide may be expressed constitutively by the cell or under inducing conditions, such as during exposure to a selected temperature or a chemical agent, such as IPTG). The polypeptide is then isolated from the cultured cells and purified, e.g., by lysing the cells (e.g., with lysozyme, exposure to a detergent, by sonication, or by some other method), fractionating cellular components, and selecting for fractions of these components which have any of: a desired enzymatic activity, immunological activity, physical characteristics (e.g., molecular mass, spectroscopic properties, and the like), and/or other biological activity.

Fractionating can be performed using affinity column chromatography where an antibody is available for a polypeptide/antigen of interest, by size exclusion chromatography to select polypeptides within a certain size range, by ammonium sulfate precipitation, polyethylene glycol precipitation, or by using combinations of these methods. Methods of purifying recombinant polypeptides from bacterial cells are well known in the art (see, e.g., Sambrook, et al., supra, and www.protocol online.net/molbio/Protein/protein_purification.htm#Protein Extraction).

Kits

The invention further provides kits comprising room temperature stable competent cells. In one embodiment, a kit is provided comprising room temperature stable competent cells in a container for shipping which does not comprise ice or any other frozen packing material. In another embodiment of the invention, room temperature stable competent cells are packaged in a sealed pouch and optionally provided along with a desiccant. In another embodiment, the cells can be stored in a closed/sealed moisture barrier, or a rigid/sealed container in the presence of desiccant. A variety of desiccants can be used to reduce the water content of the cells, including, but not limited to, calcium sulfate, silica, certain clays, polyacrylic acid, and derivatives thereof.

In a further embodiment of the invention, cells are provided along with a sample of plasmid DNA (e.g., such as lyophilized and/or supercoiled plasmid DNA) which serves as a control to monitor the transformation efficiency of the competent cells. Additional reagents can also be provided for use in transforming the competent cells, such as a substrate for a marker enzyme which is expressed by a nucleic acid to be transformed (e.g., X-Gal), rich medium (e.g., sterile SOC), antibiotics, restriction enzymes to detect signature restriction sites in a cloning vector, and the like.

### EXAMPLES

The invention will now be further illustrated with reference to the following examples. It will be appreciated that what follows is by way of example only and that modifications to detail may be made while still falling within the scope of the invention.

### Example 1

### Making Storage-Stable Competent Cells

Cell growth: E. coli XL Blue MRF cells or XL10 Gold cells, in SOB medium containing Mg salt are grown in 100 ml of LB medium supplemented to 370 mM NaCl (i.e., 200 mM higher salt than standard LB) and containing 0.25% maltose. Cells are grown at 37° C. with vigorous aeration to OD₅₅₀ of 0.45-0.5 and then placed on ice for 15-30 minutes. Cells are transferred to four chilled round- or flat-bottomed centrifuge tubes and centrifuged for 5 minutes at 5000 RPM, 4° C.

Competence induction: Cell pellets are resuspended in ¼ their original culture volume (e.g., 25 ml for 100 ml original volume) of competence-inducing buffer (e.g., FSB without glycerol, supplemented with 20% fructose or 20% sorbitol, 5% DMSO, 2 mg/ml each of proline and threonine. The volume of the competence medium is 1/4 of the original culture volume of cells (25 ml/100 ml starting volume). Cells are then incubated on ice for 18 minutes to induce competence, and harvested by centrifugation.

Pre-treatment with reducing sugar: If no additional sugar (e.g. sorbitol, fructose) was added in the competence medium, then cells are resuspended gently in ice cold 20% fructose solution in a total volume of 250 μl (100 μl/40 ml original culture volume) and incubated on ice for 10 minutes.

Desiccation: The pre-treated cells are then resuspended in a final volume of 1/40 (1 ml for every 40 ml of starting culture volume) of the desiccation medium. Cells suspended in desiccation medium are then aliquotted, at 150 μl/aliquot, into 1 or 2 ml amber flat-bottomed 13 mm diameter centrifuge vials and placed into a pre-warmed (25-30° C.) lyophilizer chamber.

The lyophilizer chamber is flushed for 2 minutes with argon, and then vacuum and increased temperature are applied as follows: 2000 mTorr at 30° C. for 30 minutes; and 1500-1200 mTorr at 30° C. for 12-18 hours or until dry. Secondary drying can then be performed at a higher temperature of approximately 40° to 45° C., for 4-5 hours, under absolute vacuum. Vials are stoppered under absolute vacuum with baked stoppers. The vials are then sealed into foil pouches, or light-tight cardboard boxes. If samples are dried in screw cap vials (and packaged without vacuum sealed), the samples can be dried further for 1 day to 1 week in a desiccator, and packaged into foil pouches containing oxygen scrubbers and desiccant.

### Example 2

### Transformation of Storage-Stable Chemically Competent Cells

Storage-stable competent cells prepared as in Example 1 are re-hydrated and transformed as follows. First, a volume of ice-cold rehydration buffer equal to the original aliquot volume (e.g., 150 μl) is added to the cells. Suitable rehydration buffers useful in the present invention include FSB (with or without 2.5×DMSO), a solution containing 10% Rubidium chloride, TFB1 broth (potassium acetate 30 mM, ribidium chloride 100 mM, calcium chloride 10 mM, manganese chloride 50 mM, final pH=5.8% or Tfb II broth (MOPS 10 mM, CaCl₂ 75 mM, rubidium chloride 10 mM, 3% fructose, final pH 6.5).

A sample of nucleic acid (e.g., about 0.01 ng-10 ng of plasmid DNA) is added to an aliquot of the re-constituted cells and the mixture is gently mixed, followed by incubation on ice for 20 to 30 minutes. Cells are then heat-shocked at 42° C. for 30-60 seconds. Following heat shock, rich medium (e.g., 1 ml of N2Y+ per 50 μl of competent cell suspension) without selective agents is added. The cell mixture is then incubated for 1 hour at 37° C. with agitation, and finally plated onto the surface of an agar plate containing medium (e.g., LB) supplemented with one or more selective agents (e.g., ampicillin) selected in accord with the vector used to transform the cells. Plates are incubated at 37° C. overnight or until colonies are visible. Single colonies are picked and analyzed or expanded as desired.

### Example 3

### Effect of Additional Sodium Chloride in Growth Medium on Desiccation Tolerance, and Transformation Efficiency (TE).

To assess the effect of additional sodium chloride in growth medium on desiccation tolerance and transformation efficiency (TE), 1 ml aliquots of frozen-thawed XL10 Gold-CamR cells were inoculated into the different growth media, which included: 1) SOB/Mg++, 2) SOB/Mg++ +100 mM NaCl, 3) SOB/Mg++ +200 mM NaCl, or 4) SOB/Mg++ 300 mM NaCl. All samples were made competent in FSB without glycerol containing 10% Fructose. The samples were desiccated in 10% trehalose and 10% sucrose, for 21 hrs, 200 m Torrs, at 30° C., with “desiccated” air (t.h.e. beads in inlet attachment) at a final cell concentration of 40:1 (150 ul aliquots), in small clear screw cap vials, or large crimp vials. Following desiccation, the vials were packed in foil pouches with desiccant beads. In some cases, desiccant beads were also added within the vials. The harvest OD550 of this prep was ˜0.6 for all samples. The results are shown in Table 1.

TABLE 1

| Sample | Growth medium | TE Dry | TE Froz |
|---|---|---|---|
| 1 | SOB/Mg only | <10⁵ | 1.7 × 10⁵ |
| 2 | 1 + 100 mM NaCl | 1.5 × 10⁶ | 4.4 × 10⁵ |
| 3 | 1 + 200 mM NaCl | 2.37 × 10⁶ | 5.4 × 10⁵ |
| 4 | 1 + 300 mM NaCl | 3.9 × 10⁶ | 4.6 × 10⁵ |

### Example 4

### Enhancement of Long-Term Stability of Room Temperature Competent Cells

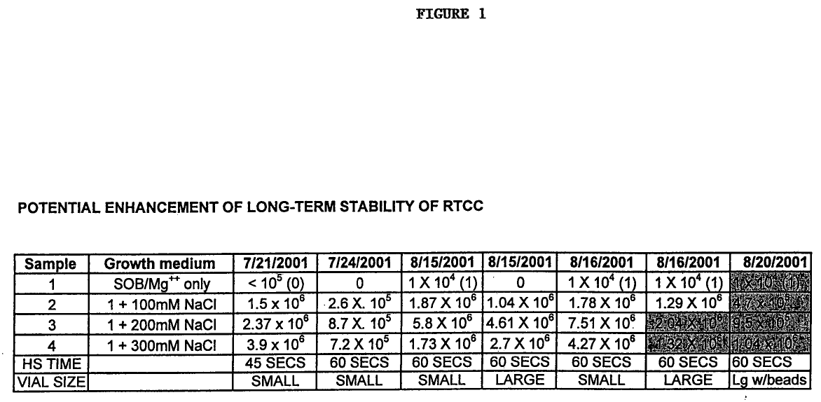

The effect of additional sodium chloride in growth medium on long-term stability of room temperature competent cells was tested. Competent cells were prepared as in Example 3. Cells were transformed with Ampicillin (AMP) resistant plasmids and plated onto LB/Amp bacterial plates at different dates throughout a one month period. The number of transformants were counted and compared. Samples were checked for background by plating mock transformations, and no background was detected. The results are shown in FIG. 1.

### Example 5

### Variations in Desiccation Medium, Competence Medium, and Growth Medium: Effect on Transformation Efficiency and Survival of Room Temperature Competent XL10Gold (CamR) Cells

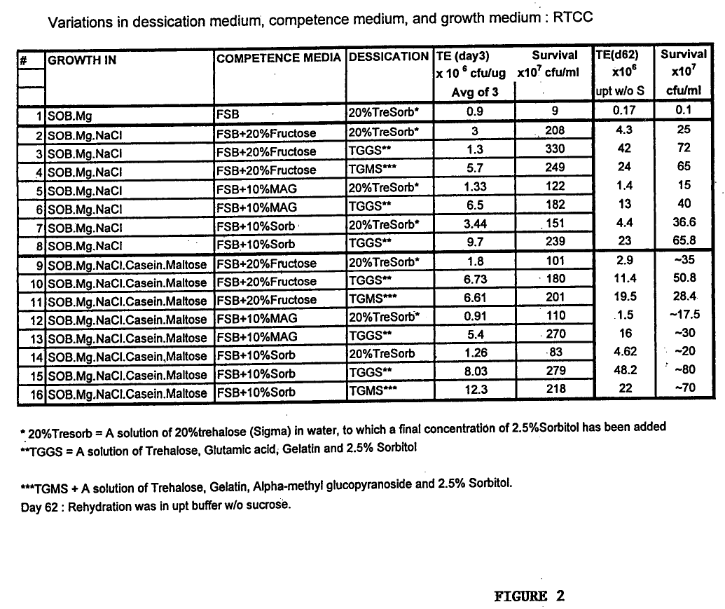

Varied desiccation medium, competence medium, and growth medium were tested for their effect on transformation efficiency and survival of room temperature competent XL10Gold (CamR) cells. For cell growth, 2 mls of thawed XL10Gold (CamR) competent cells were added to each 250 mls of growth medium, and grown at 37° C. to a final OD550 of 0.48. Cultures were chilled on ice for ˜30 mins and then harvested by centrifugation at 1.6 K RPM for 7 mins, at 4° C., with the brake set to 5. Supernatants were discarded, and excess supernatant was wiped off with paper wipes. Competence was induced by resuspending the cell pellets in FSB without glycerol containing additives such as 20% Fructose, or Sorbitol, MAG etc. After 18 min, samples were centrifuged as before, and immediately resuspended (40:1 final concentration) in desiccation media. Samples were immediately aliquoted into clear 1 ml crimp glass vials prechilled at −20° C. (150 ul per vial), in cardboard separators. Drying was done at 1500 m Torrs for 18 hr at a temperature of 30° C. in Lyostar directly on steel shelves. The samples were then pulled out of the Lyostar, capped lightly with pre-baked rubber stoppers, and dried under vacuum in the Vertis for another 2 hrs. The samples were stoppered under vacuum, and crimped under atmospheric pressure, manually. Vials were stored in a foil pouch to avoid light exposure. Transformation efficiency and survival of the room temperature competent cells was then determined. The results are shown in FIG. 2.

### Example 6

### Variations in Desiccation Medium, Competence Medium, and Growth Medium: Effect on Transformation Efficiency of Room Temperature Competent XL10Gold (Cam) Cells

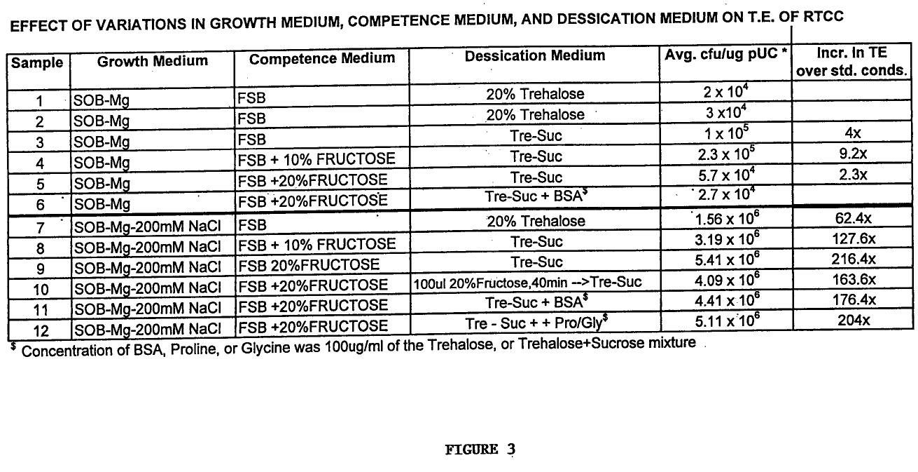

Varied desiccation medium, competence medium, and growth medium were tested for their effect on transformation efficiency of room temperature competent XL10Gold (Cam) cells. For growth, 1 ml of XL10 Gold (Cam) competent (frozen) cells were inoculated into 250 ml of growth medium, in 500 ml Corning Erlenmeyer flasks. The cells were grown for ˜2-2½ hrs at 37° C. and harvested at an OD550=0.5. Cells were chilled on ice ˜1 hr and spun down for 7 mins, at 1600 RPM, 4° C. The cells were made competent for 18 mins, with FSB (with or without additives) and spun down as before. The cell pellet was resuspended in desiccation medium and immediately aliquoted (150 ul) into 2 ml screw cap amber tubes. Drying Conditions were: 3000 m Torrs and 30° C. Transformation efficiencies were assessed 2 days after the prep was made. 2 vials from each sample (stored at room temperature in thin foil pouches containing t.h.e. desiccant beads) were resuspended in ˜175 ul chilled FSB, the contents of the two tubes were mixed, and aliquoted into a pre-chilled 2059. Supercoiled pUC was then added to the cells (0.3 ng/˜300 ul cells), mixed well, and 100 ul of the mixture aliquoted into 3 pre-chilled 2059 tubes. After incubation on ice for 25 mins, the cells were heat-shocked for 60 secs at 42° C.; 500 ul of NZY⁺ was added to each tube, and samples outgrown for 1 hour, in a 37° C. shaker. Each sample was plated out on 2 LB/Ampicillin plates, spread with beads, incubated O/N at 37° C. The results are shown in FIG. 3.

### Example 7

### Relative Survival of Chemicompetent Mutant B71 (sc19) When Dried in Trehalose or Sucrose

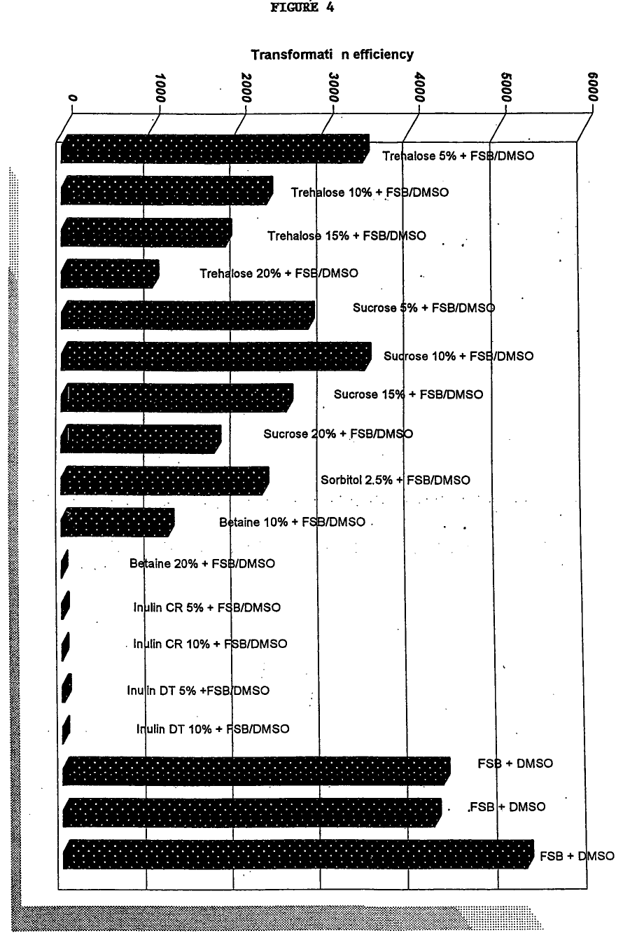

The relative survival of chemicompetent mutant B71 (sc19) when dried in trehalose or sucrose was tested. Chemicompetent mutant B71 (sc19) samples were dried in varying concentrations of Sucrose, Trehalose, Sorbitol, Betaine, Inulin, or simply FSB and DMSO (−control). The cells were harvested at an OD550=0.6 and drying was done in 2 ml glass screw cap vials; 2000 m Torrs, 30° C. FIG. 4 shows the relative transformation efficiency (TE) of the cells. Cells dried in 10% Sucrose had the highest transformation efficiency, approximately 10-fold higher than the transformation efficiency of Trehalose. To assess the relative survival, samples were rehydrated and diluted prior to plating, and colony forming units (CFU) were counted, see Table 2. The samples that were dried in varying concentrations of Sorbitol, Betaine, Inulin, or simply FSB and DMSO (−control) gave very low survival, and thus have not been included in Table 2.

An identical study with XL10Gold (Cam) gives very similar data, that is, a higher survival at ˜9.5%-to 12.5% trehalose, and correspondingly higher transformation efficiency (TE) at these concentrations. Similarly, with desiccation in Sucrose alone, XL10Gold was seen to give optimal survival/TE numbers at ˜10 to 12.5% Sucrose.

TABLE 2

| Dried in | Cfu at ⁻4 dilution | Cfu at ⁻5 dilution |
|---|---|---|
| Trehalose 5% | 618 | 65 |
| Trehalose 10% | 562 | 44 |
| Trehalose 15% | TMTC | 744 |
| Trehalose 20% | TMTC | 395 |
| Sucrose 5% | TMTC | 351 |
| Sucrose 10% * | TMTC | 458 |
| Sucrose 15% | TMTC | 820 |
| Sucrose 20% | TMTC | 896 |

### Example 8

### Varied Conditions for Generating Chemicompetent Room Temperature Competent Cells

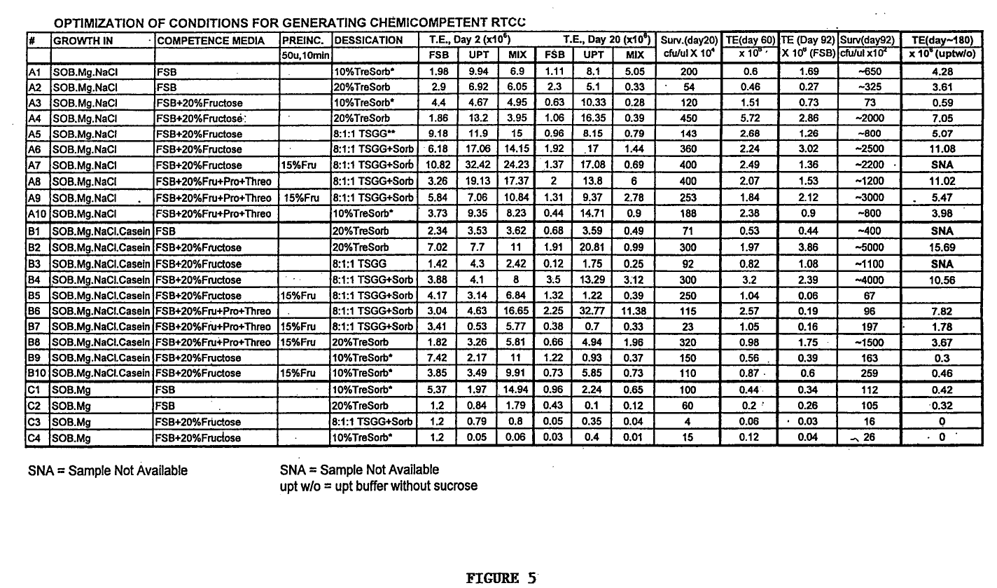

Varied conditions, including the use of fructose and/or proline and threonine in growth media, were tested for their effect on the transformation efficiency (TE) of chemicompetent room temperature competent cells. For growth, 2 mls of thawed XL10Gold comp cells were added to each 250 mls of growth medium, and grown at 37° C. to a final OD550 of 0.48. Cultures were chilled on ice for ˜30 mins and then harvested by centrifugation at 1.6 K RPM, for 7 mins, at 4° C., using a brake of 5. Supernatants were discarded, and excess supernatant was wiped off with paper wipes. Competence was induced by resuspending the cell pellets in FSB without glycerol containing additives such as 20% Fructose, or Proline, Threonine, or no additives. After 18 mins, cells were centrifuged again and the cell pellets resuspended (40:1) in desiccation media, and immediately aliquoted into clear 1 ml crimp glass vials prechilled at −20° C. (150 ul per vial), in the blue plastic holders. Samples were dried as follows: 2000 m Torrs for 30 mins and 1500 m Torrs for 18 hrs, in the Lyostar. The samples were pulled out of the Lyostar, capped lightly with pre-baked rubber stoppers, and dried under vacuum in the Vertis for another 2 hrs. The samples were stoppered under vacuum and crimped under atmospheric pressure, manually. Vials were stored in a foil pouch to avoid light exposure. The transformation efficiency and survival was assessed as previously described, the results of which are presented in FIG. 5.

All of the references identified hereinabove, are hereby expressly incorporated herein by reference to the extent that they describe, set forth, provide a basis for or enable compositions and/or methods which may be important to the practice of one or more embodiments of the present inventions.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims:

1. A method of generating storage-stable competent cells, said method comprising:
a) growing bacterial cells in culture medium at hyperosmotic salt concentration;
b) treating said cells to make them competent;
c) contacting said cells with a solution comprising a reducing sugar or a non-reducing sugar, or both; and
d) drying the competent cells resulting from step (c) in the presence of a non-reducing sugar, such that storage-stable competent cells are generated.

2. The method of claim 1 wherein said salt is NaCl.

3. The method of claim 1 wherein said hyperosmotic salt concentration is 100 mM to 350 mM above isoosmotic.

4. The method of claim 1 wherein said hyperosmotic concentration of NaCl is 150 mM to 225 mM above isoosmotic.

5. The method of claim 1 wherein said hyperosmotic salt concentration is 200 mM above isoosmotic.

6. The method of claim 1 wherein step (c) is performed either during or after step (b), but before step (d).

7. The method of claim 1 wherein said drying step is performed at a temperature above freezing.

8. The method of claim 1 wherein step (d) comprises drying said cells in the presence of a non-reducing sugar selected from the group consisting of trehalose, sucrose, α-methyl glucopyranoside, α-methyl galactopyranoside, and sorbitol.

9. The method of claim 1 wherein step (c) comprises contacting said cells with a non-reducing sugar selected from the group consisting of trehalose, sucrose, α-methyl glucopyranoside, α-methyl galactopyranoside, and sorbitol.

10. The method of claim 1 wherein step (c) comprises contacting said cells with a reducing sugar selected from the group consisting of fructose, glucose (dextrose), maltose, lactose, glucopyranose, ribose and cellobiose.

11. The method of claim 9 wherein step (c) comprises contacting said cells with a non-reducing sugar selected from sorbitol and α-methyl glucopyranoside.

12. The method of claim 10 wherein step (c) comprises contacting said cells with a reducing sugar, wherein said reducing sugar is fructose.

13. The method of claim 1 wherein step (c) comprises contacting said cells with fructose and a non-reducing sugar selected from sorbitol or α-methyl glucopyranoside.

14. The method of claim 10 wherein in step (c), or step (d) said reducing sugar and said non-reducing sugar is present at a total sugar concentration of 10-25% (w/v).

15. The method of claim 1 wherein said cells are made competent by exposure to a chemical agent.

16. The method of claim 15 wherein said chemical agent is selected from the group consisting of CaCl₂, RbCl₂, MnCl₂, and hexamine cobalt chloride.

17. The method of claim 1 wherein said step of drying the competent cells is performed under vacuum.

18. The method of claim 17 wherein said step of drying the competent cells is performed at a temperature above freezing.

19. The method of claim 1 step (a) comprises growing said bacterial cells to a final OD550 of 0.45 to 0.5.

20. The method of claim 1 wherein said bacterial cells are Gram negative cells.

21. The method of claim 1 wherein said culture medium comprises casein hydrolysate and/or maltose.

22. The method of claim 21 wherein said casein hydrolysate is present in said culture medium at a concentration of 11-15 g/liter.

23. The method of claim 21 wherein said casein hydrolysate is present in said culture medium at a concentration of 11-12 g/liter, inclusive.

24. The method of claim 21 wherein said maltose is present in said culture medium at a concentration of 0.1-0.3% (w/v).

25. The method of claim 21 wherein said maltose is present in said culture medium at a concentration of 0.2-0.3% (w/v), inclusive.

26. The method of claim 1 wherein said step of treating cells to make them competent comprises contacting said cells with a defined solution comprising one or both of proline and threonine.

27. The method of claim 26 wherein said defined solution comprises proline, threonine or both at a concentration of 0.5-7.5 mg/ml.

28. The method of claim 26 wherein the concentration of proline, threonine or both in said defined solution is from 2-4 mg/ml, inclusive.

29. The method of claim 1 wherein said competent cells can be stored at temperatures above −80° C. for at least one month and maintain transformation efficiencies of at least 105 colonies/μg DNA.

30. The method of claim 1 wherein said competent cells can be stored at temperatures of −20° C. or above for at least one month and maintain transformation efficiencies of at least 105 colonies/μg DNA.

31. The method of claim 1 wherein said competent cells can be stored at temperatures of 0° C. or above for at least one month and maintain transformation efficiencies of at least 105 colonies/μg DNA.

32. The method of claim 1 wherein said competent cells can be stored at temperatures of 4° C. or above for at least one month and maintain transformation efficiencies of at least 105 colonies/μg DNA.

33. The method of claim 1 wherein said competent cells can be stored at temperatures of 15° C. or above for at least one month and maintain transformation efficiencies of at least 10⁵ colonies/μg DNA.

34. The method of claim 1 wherein said competent cells can be stored at temperatures of 20° C. or above for at least one month and maintain transformation efficiencies of at least 10⁵ colonies/μg DNA.

35. The method of claim 1 further comprising the step, during or after step (c), of limiting the exposure of said competent cells to oxygen.

36. The method of claim 35, comprising the step, after step (c), of storing said competent cells in a vacuum stoppered vial.

37. The method of claim 36, comprising the step of storing said vial in a sealed pouch.

38. The method of claim 35 wherein said limiting comprises drying and/or storing said competent cells in the presence of an oxygen scavenger.

39. The method of claim 1 further comprising the step, after step (c) of limiting exposure of said competent cells to moisture.

40. The method of claim 39 wherein the stopper in said stoppered vial is baked to remove moisture prior to use.

41. The method of claim 1 further comprising the step, during or after step (c), of limiting the exposure of said competent cells to light.

42. The method of claim 41 wherein said limiting comprises storing said competent cells in a vial that has reduced transmittance of light.

43. A preparation of storage stable competent cells prepared according to claim 1.

44. A kit comprising the preparation of claim 43.

45. The method of claim 1 wherein step (d) comprises drying the competent cells resulting from step (c) in the presence of a non-reducing sugar and gelatin, such that storage-stable competent cells are generated.

46. The method of claim 45 wherein said gelatin is present at 0.5 to 2.5%

47. The method of claim 45 wherein said gelatin is present at 0.8 to 1.2%.

48. A method of producing a transformed cell, said method comprising
a) obtaining cells generated according to the method of claim 1;
b) re-hydrating said cells;
c) contacting said cells with a nucleic acid vector; and
d) growing said cells, such that a transformed cell is produced.

49. A method of producing a recombinant polypeptide comprising:
a) obtaining cells generated according to the method of claim 1;
b) rehydrating said cells;
c) contacting the rehydrated cells with a nucleic acid encoding said recombinant polypeptide; and
d) growing said cells in a cell growth medium under conditions in which the cells produce said polypeptide.

50. The method of claim 48, in which cells which have taken up said nucleic acid are separated from cells which have not taken up said nucleic acids.

51. The method of claim 49, wherein said recombinant polypeptide is isolated from said cells.
